(19) United States
(12) Patent Application Publication
(10) Pub. No.: US 2021/0236677 A1
(43) Pub. Date: Aug. 5, 2021

(54) STERILIZATION MONITOR

(71) Applicant: TUTTNAUER LTD., BEIT SHEMESH (IL)

(72) Inventor: Boaz NITZAN, Sde Warburg (IL)

(21) Appl. No.: 17/052,546

(22) PCT Filed: May 7, 2019
(86) PCT No.: PCT/IB2019/053737

§ 371 Date: Nov. 3, 2020

Related U.S. Application Data
(60) Provisional application No. 62/672,597, filed on May 17, 2018.

(51) Int. Cl. A61L 2/28; A61L 2/24; A61L 2/06; C12Q 1/22

(52) U.S. Cl. CPC A61L 2/28; A61L 2/24; A61L 2/06; C12Q 1/22; G01N 2333/33; A61L 2202/122; A61L 2202/24; G01N 2333/32; A61L 2202/14

(57) ABSTRACT

The present invention provides a system for monitoring a sterilization procedure. The system includes a sterilization unit for providing sterilization of at least one item, a biological indicator for producing a readable signal, the signal readable during the sterilization procedure, wherein the signal corresponds to the viability of the biological indicator and a reader for reading the signal from the biological indicator during the sterilization procedure for monitoring the sterilization real time. Furthermore, the present invention provides a method for monitoring sterilization featuring simultaneously exposing at least one item to be sterilized and a biological indicator to a sterilization medium, the biological indicator producing a measurable signal corresponding to its viability and simultaneously monitoring the signal from the biological indicator during the exposure to the sterilization medium to determine completion of effective sterilization of the at least one item.

This application claims priority to U.S. patent application Ser. No. 62/672,597 filed on May 17, 2018 and incorporated by reference herein in its entirety.

### FIELD OF THE INVENTION

The present invention relates to a sterilization monitor. Moreover, the present invention is of a real time sterilization indicator and use thereof in a sterilizer device.

### BACKGROUND OF THE INVENTION

Sterilization is carried out routinely in many industries and on diverse items, such as medical solutions, equipment, devices and food and beverages. In order to ascertain that the sterilization has been successful in eliminating all microorganisms, a sterilization indicator may be employed. Sterilization cycle efficacy tests are commonly done post the sterilization process using sterilization indicators, which include biological, enzyme and chemical indicators.

Chemical indicators give immediate visible results, such as change of color, which indicates that the items in the sterilizer have been exposed to a certain temperature for a certain period of time, but this is not an accurate indicator of successful sterilization.

Biological indicators are more accurate monitors of sterilization. One type of common indicator uses a known quantity of test microorganisms. The test microorganisms are placed in a carrier near the items to be sterilized and exposed to the sterilization procedure. After sterilization the spores are incubated in a growth medium and spore outgrowth is monitored. This method is limited in that the need for post incubation means that the monitoring is not real time. With some of these types of indicators the incubation time is lengthy and can be for a period of more than 24 hours. Such a time period is not conducive to daily multiple use of a piece of sterile equipment. Further, in view that this method is not a real time assessment of the sterilization, in some instances the calculated sterilization cycle's time may be substantially longer than is actually needed. This unnecessarily delays use of items, which are sterile.

Enzyme indicators emit light which can be measured with a luminometer resulting in measurable data. This may be faster than biological indicators. However, these indicators are indirect indicators of sterilization and assume that the rate of enzyme inactivation is the same as the rate of killing spores. This assumption is not reliable. The amount of enzyme needs to be carefully calibrated and calibration errors may affect the reliability of results.

It would therefore be desirable to have a sterilization indicator and method of use thereof which enables reliable real time monitoring of a sterilization procedure. It would be valuable to have an indicator and method of use which is directly proportional to the killing rate of spores. Furthermore, it would be advantageous if sterilization time was controlled according to the reading of a real time monitor. It would also be beneficial for the sterilization indicator to be suitable for use with different sterilization procedures. The present invention provides such a real time monitor, indicator and methods of use.

### SUMMARY

The invention may have several aspects. One aspect is a system for monitoring a sterilization procedure. The system may include a sterilization unit for providing sterilization of at least one item. The system may include a biological indicator for producing a readable signal, the signal readable during the sterilization procedure and the signal corresponding to viability of the biological indicator. The system may include a reader for reading the signal from the biological indicator during the sterilization procedure for monitoring the sterilization real time.

In various embodiments the sterilization unit may feature a window, through which the signal can be detected and read by the reader. The sterilization unit may include a compartment for holding the biological indicator and wherein the compartment is constructed and positioned to facilitate reading of the signal from the biological indicator through the window. The reader may be adjacent to the window, for detecting and reading the signal from the biological indicator, through the window. The sterilization unit may be an autoclave. The system may be a system for monitoring sterilization real time. The system may be for monitoring sterilization during the sterilization process. The biological indicator may feature a microorganism including at least one of Bacillus, Geobacillus and Clostridia species of microorganisms. The biological indicator may include at least one of dormant microorganisms for producing a readable signal and activated microorganisms for producing a readable signal, the signal corresponding to viability of the biological indicator. The biological indicator may produce a signal relating to at least one of chemiluminogenicity, bioluminogenicity, fluorogenicity, chromogenicity, electrochemiluminescence, photoluminescence, phosphorescence, thermoluminescence, potential difference, magnetism and radiation. The readable signal may be potential difference. The readable signal may be magnetic. The microorganism may be manipulated to exhibit bioluminescence. The microorganism may be manipulated to exhibit magnetism. A measured signal may indicate that microorganisms are viable. A measured signal, which is higher than a signal equivalent to the sterility assurance level may indicate that the sterilization of the at least one item is not complete. No signal may indicate that substantially all the microorganisms are dead. No signal, or a signal equal to about the sterility assurance level or a signal equal to an amount of viable microorganisms lower than the sterility assurance level, may indicate that the sterilization of the at least one item is complete. The sterilization procedure may employ a sterilization medium for hot sterilization or cold sterilization. The hot sterilization may employ at least one of dry heat and steam. The cold sterilization may employ at least one of radiation, plasma, a gaseous sterilant and a liquid sterilant. The reader may feature a sensor. The sensor may be at least one of an optical sensor, an electrical sensor and a magnetic sensor. The reader may include a display for displaying the progress of a sterilization procedure. The reader may be calibrated to translate a signal from the biological indicator into an amount of viable microorganisms present in the biological indicator. The reader may verify that the at least one item is sterile. The reader may be connected to the sterilization unit. The sterilization unit may include a controller for controlling the sterilization procedure. The controller may be connected to the reader. The controller may be electrically connected to the reader. The controller may turn off the sterilization cycle as a result of the reader receiving and reading no signal or a signal equal to about the sterility assurance level or lower from the biological indicator. The biological indicator may be covered by a substrate and wherein the substrate is permeable to the signal. The at least one item may be at least one of medical equipment, medical device, surgical equipment, dental equipment, cosmetic product, cosmetic equipment, hair cutting equipment, food, beverage, a pharmaceutical product, a solution, packaging and a recycled product.

A further aspect is a method of monitoring sterilization. The method may include simultaneously exposing at least one item to be sterilized and a biological indicator to a sterilization medium, the biological indicator producing a measurable signal corresponding to its viability. The method may include simultaneously monitoring the signal from the biological indicator during the exposure to the sterilization medium to determine completion of effective sterilization of the at least one item. The method may include placing a biological indicator including a microorganism in a sterilization unit of the system of the present invention, the sterilization unit containing at least one item to be sterilized. Placing may include placing the biological indicator so that it is readable by a reader through a window in the sterilization unit. The method may include running a sterilization cycle in the sterilization unit. Monitoring may include reading a signal from the biological indicator during the sterilization cycle. Reading a signal may be continuous. Reading a signal may be in time intervals. Reading a signal may be at the end of the sterilization cycle. Reading a signal may feature reading luminosity from the biological indicator. Reading a signal, may include reading a magnetic signal from the biological indicator. Reading a signal may include reading potential difference of the biological indicator.

In various embodiments of the method of monitoring sterilization the method may include connecting a reader to the sterilization unit. The method may include reading a signal from the biological indicator prior to the start of the sterilization cycle. The method may include determining the sterilization cycle has completed a successful sterilization according to the reading. The method may include determining the sterilization is incomplete when reading a signal detects and reads a signal indicative of an amount of microorganisms higher than the sterility assurance level from the biological indicator. The method may include determining the sterilization is complete when reading a signal reads there is no signal, or a signal substantially equivalent to the sterility assurance level or lower, from the biological indicator. The method may include determining the at least one item is sterile when reading a signal reads there is no signal, or a signal substantially equivalent to or lower than the sterility assurance level from the biological indicator. The method may include stopping the sterilization process according to the determining from the signal when the sterilization is successfully completed. Stopping the sterilization process may feature the reader communicating with a sterilization unit controller when the reader detects no signal from the biological indicator or a signal equal to or lower than about the sterility assurance level. Stopping the sterilization process may include the controller turning off the sterilization process as a result of the communication of no signal or a signal equal to about or lower than the sterility assurance level.

A still further aspect is a method of sterilization. The method may feature placing a biological indicator including a microorganism in a sterilization unit, the sterilization unit containing at least one item to be sterilized. The method may include running a sterilization cycle in the sterilization unit. The method may include reading a signal with a reader from the biological indicator during the sterilization cycle. The method may include determining from the signal when the sterilization is successfully completed. The method may include stopping the sterilization process according to the determining from the signal when the sterilization is successfully completed. Stopping the sterilization process may include the reader communicating with a sterilization unit controller when the reader detects no signal from the biological indicator or a signal equal to or lower than about the sterility assurance level and the controller turning off the sterilization process as a result of the communication of no signal or a signal equal to or lower than about the sterility assurance level.

An additional aspect is a method of validating that items are sterile after a sterilization cycle. The method may include placing a test biological indicator comprising a microorganism in a sterilization unit, the sterilization unit containing at least one item to be sterilized. The method may include running a sterilization cycle in the sterilization unit. The method may include reading a signal with a reader from the test biological indicator after completion of the sterilization cycle. Reading a signal may include validating that the at least one item is sterile when reading that there is no signal or a signal substantially equivalent to or lower than the sterility assurance level from the biological indicator.

An aspect is a sterilization device for monitoring a sterilization procedure. The device may include a sterilization unit for providing sterilization of at least one item. The device may include a reader connected to the sterilization unit, the reader for reading the signal from the biological indicator, wherein the reader is adjacent to a window of the sterilization unit, for detecting and reading the signal from the biological indicator through the window. The sterilization unit may include an internal chamber for placing the at least one item within to be sterilized. The sterilization unit may include a compartment in the internal chamber for holding a biological indicator, the biological indicator producing a readable signal, the signal corresponding to viability of the biological indicator. The sterilization unit may include a window, through which a signal from the biological indicator can be detected and read by the reader.

In various embodiments of the sterilization device the reader may be calibrated for translating a signal from the biological indicator to an amount of viable microorganisms present in the biological indicator. The reader may be calibrated for translating a signal from the biological indicator to verification of a complete or incomplete sterilization of the at least one item. The compartment may be constructed and positioned to facilitate reading of a signal from the biological indicator through the window of the sterilization unit.

### BRIEF DESCRIPTION OF THE DRAWINGS

The various features of the invention will best be appreciated by simultaneous reference to the description which follows and the accompanying drawings, which are not drawn to scale and in which:

FIG. 1 shows a schematic view of an exemplary sterilization monitoring system according to an aspect of the present invention;

FIG. 2 shows a schematic view of an exemplary sterilization monitoring system according to an aspect of the present invention;

FIG. 3 shows a schematic view of an exemplary sterilization monitoring system according to an aspect of the present invention;

FIG. 4 shows a flow chart of an exemplary method of use of the system to monitor sterilization according to an aspect of the present invention;

FIG. 5 shows a flow chart of an exemplary method of use of the system to monitor sterilization according to an aspect of the present invention;

FIG. 6 shows a flow chart of an exemplary method of automated sterilization cycle time using a sterilization indicator according to an aspect of the present invention;

FIG. 7 shows a flow chart of an exemplary method of use of the system to verify sterilization according to an aspect of the present invention; and

FIG. 8 shows a flow chart of an exemplary method of production of a sterilizing monitoring system according to an aspect of the present invention.

### DETAILED DESCRIPTION

In one aspect the present invention is of a sterilization system. The system may be a system for monitoring a sterilization procedure. It is envisioned that the present invention may be used in a variety of industries, which rely on sterile products, such as medical, pharmaceutical, food, drink and recycling industries. In an additional aspect the present invention is of a method of monitoring a sterilization cycle. Further, the present invention provides a system for validating sterility of items.

The system of the present invention is relatively facile to use and facilitates a real time monitoring of sterility, without the need for lengthy post sterilization incubation. The system of the present invention facilitates a sterilization process where the time for completion does not include the addition of post cycle waiting time or even in some cases unneeded time of part of the set sterilization cycle. As such, the time for sterilization, which includes verification of sterilization using the system of the present invention may range from about fifteen minutes to about eighty minutes. Accordingly, the present invention enables more frequent repeated use of sterile equipment. This is especially advantageous where equipment is limited and routinely needed. In addition, the system and method of the present invention facilitate following the status of the sterilization including identifying stages before sterilization has been achieved, such as disinfection. This enables control of the extent of sterilization and the ability to stop the sterilization cycle when a desired end point has been reached.

As used herein the term ‘readable’ may include, but is not limited to a signal which can be read by a reader. The term includes a signal which can be detected by a reader. The term includes a signal, which can be measured by a reader.

As used herein the term ‘sterilization’ may include, but is not limited to elimination, killing, removal or deactivation of biological agents, such as, but not limited to microorganisms, pathogens, bacteria, viruses, fungi, spore forms and prions from a specified region. Sterilization may be performed using a sterilization process and may run for longer than is required to provide a sterility assurance level. The term may include a process providing a sterility assurance level of at least 10⁻⁶.

As used herein the term ‘disinfection’ may include, but is not limited to a process to destroy microorganisms on a specified region. The term includes a method, which is less effective than sterilization at killing microorganisms and may not kill all microorganisms on a specified area, such as resistant bacterial spores.

As used herein the terms ‘a’ and ‘an’ may mean ‘one’ or ‘more than one’.

As used herein the terms ‘comprising’, ‘including’, ‘containing’, ‘featuring’, ‘having’ and any fauns of the terms thereof are inclusive and open ended and do not exclude additional, elements or methods steps, which are not recited.

The principles and operation of a system and device, such as a sterilization system and device and methods of use thereof according to the present invention may be better understood with reference to the figures. The figures show non-limiting aspects of the present invention.

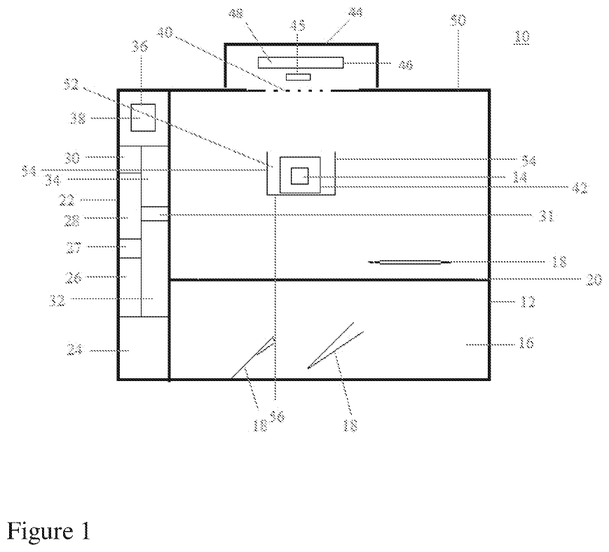

FIG. 1 shows schematic views of a system 10 according to an aspect of the present invention. In one aspect the system 10 may include a sterilization device 12 and a biological indicator 14.

The sterilization device 12 may be any suitable device for carrying out sterilization, such as an autoclave. Any type of autoclave may be used. The sterilization device 12 may use any type of sterilization technology or medium including hot or cold sterilization. Other non-limiting examples of sterilization devices 12 include dry heat sterilizers and sterilization devices, which employ radiation. Hot sterilization may employ, for example at least one of dry heat and steam. Cold sterilization may employ, for example at least one of radiation, plasma, a gaseous sterilant and a liquid sterilant. Non limiting examples of gaseous and liquid sterilants include hydrogen peroxide, ethylene oxide, formaldehyde, peracetic acid, nitrogen dioxide, ozone and mixtures thereof. The sterilization device 12 may include an internal space 16, such as a chamber 16 for placing items 18 to be sterilized. The sterilization device 12 may include at least one shelf 20. The at least one shelf 20 may be used for placing on the at least one shelf, equipment or items 18 to be sterilized. The device 12 may include a control 22. The control 22 may be for controlling the sterilization process. The control 22 may include any suitable control related features. In some embodiments, the control 22 may include preprogrammed sterilization cycles 24, a temperature control 26, a resistance control 27, a pressure control 28, a steam control 30, a timer 31, an on/off switch 32, and wireless communication for connection with remote devices 34. The control 22 may include a screen 36 and a display 38. The sterilization device 12 may be made from any suitable material, such as, but not limited to metal, stainless steel, high density polypropylene and glass. The material is configured to withstand the sterilization medium and sterilization conditions. The sterilization unit 12 may include a window 40. The window 40 may be constructed of a material, which does not obstruct and is permeable to a signal, allowing reading of a signal from the biological indicator, so that the signal is detectable and readable by a reader 44. The window 40 may be constructed from a material such as, but not limited to glass, quartz or any highly transparent material. The window 40 material may not substantially exhibit absorption in any luminescence wavelength, or interact with any other type of signal from a biological indicator. The material of the window 40 is constructed to withstand the sterilization conditions of the sterilization unit 12. The window 40 may be positioned in any suitable position on the sterilization unit 12, which facilitates a direct path from the window 40 to the biological indicator 14 for an unobstructed signal. In some embodiments, the window 40 is positioned on the ceiling 50 of the sterilization unit 12. The sterilization device 12 may be designed so that there is no noise to interfere with the readable signals from a biological indicator 14 used with the sterilization device 12. The sterilization device 12 may include an electrical cage to prevent interference by noise.

The biological indicator 14 may include any suitable microorganism. Suitable microorganisms may have a known sterilization resistance to the sterilization mode. Microorganisms may include suitable pathogens, spores, bacteria, virus, and fungi. In some embodiments the microorganism includes at least one of Bacillus, Geobacillus and Clostridia species of microorganisms. In one non-limiting example the microorganism is Geobacillus stearothermophilus. The microorganism may be one approved for use by the FDA or an equivalent authority. The microorganism may exhibit a signal. The dormant microorganisms may exhibit a signal. The activated microorganism/spores of the biological indicator 14 may exhibit a signal. Microorganisms, such as spores may be activated by conditions, which may include but are not limited to heat, humidity and pH. Microorganisms, such as spores may change from a dormant state to an activated state by the conditions of the sterilization unit and sterilization medium. The microorganism may exhibit a signal, which is detectable or readable or can be measured in any suitable way. The microorganism may exhibit a signal, which is readable by a reader 44. The signal may be a signal which can be graphically displayed by the reader 44 and/or the sterilization unit 12. The signal may be a signal, which is eliminated or modified by sterilization and the resultant death of the source of the signal, such as the spore or other pathogen. The signal may be a result of or related to at least one property of chemiluminogenicity, bioluminogenicity, fluorogenicity, chromogenicity, magnetism, electricity, current, charge, radiation and optical density, phosphorescence, electrochemiluminescence, photoluminescence, thermoluminescence and radiation. In some embodiments, the signal is the potential difference of the microorganism cells. The microorganism may be manipulated to exhibit a property, which produces a signal. Manipulation may include any suitable type of chemical, physical, biological, genetic or other manipulation. In some embodiments, the microorganism has been manipulated to exhibit bioluminescence, such as by genetic engineering to facilitate a luminescent signal. In some embodiments, the microorganism has been manipulated to exhibit magnetism to facilitate a magnetic signal.

The biological indicator 14 may be available in different forms. One form features spores that are added to a carrier and packaged 42 to maintain the viability of the combination. The number of spores may be any suitable amount. In one non-limiting example about 1.5 million to about 2 million spores are added to the carrier. The carrier and packaging 42 may be inert and designed to withstand the conditions of the sterilization and to allow the sterilization medium to penetrate and contact the biological indicator 14. The carrier and packaging 42 are constructed so that they do not hinder or affect the signal from the biological indicator 14 and so that the signal can be measured and read. A suitable carrier includes paper. The carrier may be of any suitable dimensions. In one non-limiting example, the carrier may be from about 0.15 mm to about 1 mm thick. The packaging may include a frame, which may be made from any suitable material such as a plastic or metal frame. The packaging 42 may include a backing such as a backing, which is permeable to the sterilization medium. The packaging 42 may include at least one transparent side, such as one of the backside, front side, left side, right side, top side and bottom side. In one non-limiting example the packaging 42 may include a transparent top side, which may be made from a material such as PET or glass. The packaging and substrate may be disposable. In one embodiment, the packaging and substrate may be reusable. An additional form of the biological indicator 14 is a spore suspension, which may be used to inoculate one of the units to be sterilized 18. A further form of the biological indicator 14 may be used in one embodiment, wherein the biological indicator 14 is packaged with a medium, such as at least one of a growth medium, germinant and nutrients.

The biological indicator 14 may be placed in the sterilization device 12. The biological indicator may be positioned so that it is exposed to substantially the same sterilization conditions as the items to be sterilized 18. The sterilization conditions, such as heat and/or moisture may activate the biological indicator microorganisms. The biological indicator 14 may be placed on a separate shelf 20 or may be placed on the same shelf or other surface as the items to be sterilized 18. The biological indicator 14 may be positioned so that it is in line with the window 40 of the sterilization device 12. The biological indicator 14 may be placed so that there is an unobstructed path to the window 40 so that a signal from the biological indicator 14 may be read by a reader 44. The sterilization unit 12 may include a compartment 52 for holding the biological indicator 14. The compartment may be in the chamber 16 of the sterilization device 12. The compartment 52 may be sized so that the biological indicator 14 fits into the compartment 52. The compartment 52 may be positioned under the window 40 or in any suitable position so that a signal permeable wall of the compartment 52 faces the window 40 and so that a signal from the biological indicator 14 when placed in the compartment 52 is in line with the window 40 and can be read by a reader 44. The compartment 52 may be constructed from side walls 54 and a bottom wall 56. The side walls 54 may include two side walls, such as a left side wall 54 and a right side wall 54. The side walls may include a back wall 54 and a front wall 54. In one embodiment, the back wall of the compartment 52 may be constructed from part of the back wall of the chamber 16. In some embodiments, the compartment 52 may not include a top wall, in order for the signal to be readable from the window 40. In some embodiments, the compartment 52 may include a top wall made from glass or another material, which is permeable to the signal and through which a signal from the biological indicator 14 could be read. In some embodiments, the compartment 52 may not include at least one wall or may include a signal permeable wall, which faces and is in line with the window of the sterilization unit in order that a signal from the biological indicator 14 is readable from the window 40. In an embodiment, wherein an item to be sterilized is inoculated with a suspension of the biological indicator, the item to be sterilized may be placed in the compartment 52 or in any other suitable position within the sterilization unit 12, so that the signal from the biological indicator 14 can be read by a reader 44. In one embodiment, the sterilization chamber 12 may not include a compartment 52. In such an embodiment without a compartment, the sterilization chamber 12 may include an area, such as a position on a shelf, which is marked for accurate placement of the biological indicator 14 by a user, in order that the biological indicator 14 is positioned for reading by a reader during the sterilization procedure.

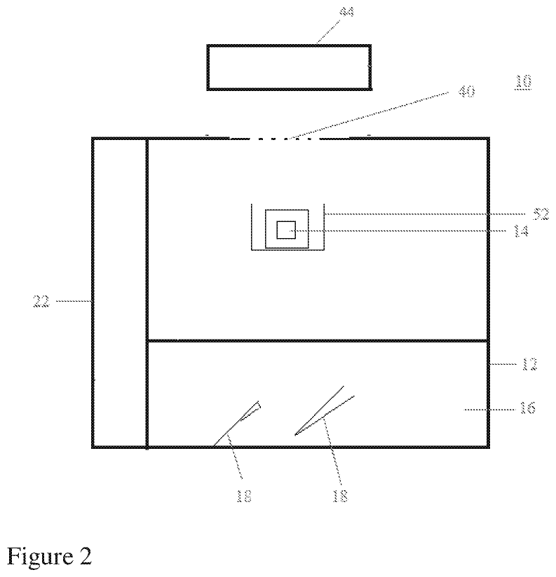

The system 10 may include a reader 44. The reader 44 may be any suitable reader 44. The reader 44 may be physically connected to the sterilization device 12 as shown in FIG. 1, such as but not limited by a mechanical and/or electrical connection. In an alternative embodiment shown in FIG. 2 the reader 44 may not be physically connected to the sterilization device 12. Physical connection may not be needed for reading certain signals, such as, but not limited to optical signals. The reader 44 may be positioned adjacent to the window 40 in the sterilization unit 12 in order that it can detect and read a signal from the biological indicator 14 through the window 40. In an embodiment wherein the window 40 is positioned on the ceiling of the sterilization unit 12 and the reader 44 is connected to the sterilization device, the reader 44 may be positioned on the ceiling of the sterilization unit 12 in communication with the window 40. In some embodiments, wherein the reader 44 is not physically connected to the sterilization unit 12, the reader 44 may be positioned at a distance from the sterilization unit 12, which still enables reading of the signal from the biological indicator 14 through the window 40.

The reader 44 may include at least one sensor 45. The sensor 45 may be positioned in the reader 44 such that it can detect and read a signal from the biological indicator 14 through the window 40. The sensor 45 may be at least one of an optical sensor, an electrical sensor, a colorimeter sensor and a magnetic sensor. In some embodiments the reader 44 may be configured to detect and read one type of signal from the biological indicator 14, such as an optical reader to detect changes in light and/or emission. In some embodiments, the reader 44 is configured to detect different types of signal from a biological indicator 14. The reader 44 may be configured to detect an applicable signal or may be configured to detect more than one signal. In one non-limiting example a reader 44 may be configured to only detect and measure potential difference. In an alternative non-limiting example the reader 44 may be configured to detect potential difference and difference in luminescence. The reader 44 may be configured to detect an applicable signal from the biological monitor 14 and to prevent interference from any noise from other sources.

The reader 44 may include a screen 46. The screen 46 may include a display 48. The display 48 may display the progress of a sterilization cycle. A signal from the biological indicator 14 may be graphically displayed by the reader 44 on the screen 46.

The reader 44 may be calibrated. The reader 44 may be calibrated for reading a type of signal as a measure of sterility. The reader 44 may be calibrated such that a sterility assurance level of at least about 10⁻⁶ or any other predetermined level is indicative that sterility has been achieved and that substantially all the biological indicator spores 14 have been killed. A signal from the biological indicator of about the sterility assurance level or a more stringent lower level of less than about 10⁻⁶ may be determined as a no signal, wherein a no signal is indicative that the contents of the sterilization unit 12 are sterile, such as for example the item/s 18 placed in the sterilization unit for sterilization are sterile. Calibration may be done by any suitable method. In one embodiment the reader may be calibrated by taking a known amount of the microorganism, such as the amount of the microorganism used in the biological indicator 14 and measuring the readable signal from the microorganism with the reader 44 to obtain a time zero reading. This may be repeated with smaller known amounts of the biological indicator, until an amount equal to the sterility assurance level. The sterility assurance level of 10⁻⁶ of the microorganism or whatever level has been determined as the sterility assurance level may be used and the signal measured therefrom with the reader to obtain the reading when sterility has been achieved. Calibration may also be done using a timer and the sterility conditions of the sterilization unit 12. Readings are done at spaced apart time intervals and the amount of microorganisms can be measured at each reading. A separate calibration may be performed for every different type of signal and for every type of microorganism being used in the biological indicator 14. The calibration may be saved to the reader 44 and may be an integrated feature of the reader. A user may not need to calibrate the system, wherein the reader 44 includes the calibration data. In some embodiments, the reader 44 may read the signals from the biological indicator 14 and the calibration and translation of the readings may be done by the control 22 of the sterilization unit 12. In such an embodiment, the calibration data is integrated in the control 22 of the sterilization unit 12.

A readable signal from the biological indicator 14, indicative of a higher level of microorganisms than the sterility assurance level, is indicative that the spores are still viable. When the reader 44 no longer detects a signal, or detects a signal indicative of the sterility assurance level or a lower level of microorganisms than the sterility assurance level, the display 48 may show no signal, an indication that sterilization has been completed and a sufficient number of, or all, the spores of the biological indicator 14 are no longer viable and have been killed. The reader 44 may continually read signals from the biological indicator 14, or may read signals from the biological indicator 14 at time intervals. The time intervals may be any time interval, such as defined by a user. The reading may be automatic or manual. In some embodiments, the reader 44 may read a signal from the biological indicator 14 before the sterilization cycle. This may provide a baseline signal of viable, live spores. The baseline signal may be of viable dormant spores, viable activated spores or a combination thereof. In some embodiments, the reader 44 may read a signal from the biological indicator 14 at the end of a sterilization cycle. No signal or a signal indicative of at least the sterility assurance level or lower, at the end of the sterilization cycle facilitates verification that the sterilization cycle has been successfully completed and the at least one item 18 in the sterilization device is sterile.

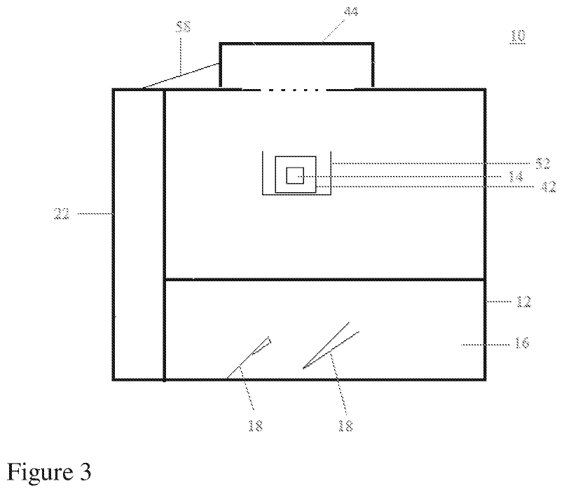

In some embodiments as shown in FIG. 3, the reader 44 may be connected to the controller 22 of the sterilization device 12 by a connection 58. The connection 58 may be any suitable connection 58. The connection 58 may be at least one of a physical connection, an electrical connection and a wireless connection. The reader 44 may be in communication with the controller 22. The controller 22 may interrogate the reader 44 in order to determine how long to continue the sterilization cycle. The reader 44 may detect and read signals from the biological indicator 14. When the reader 44 detects a signal this may be communicated to the controller 22 and sterilization is continued. When the reader 44 detects a signal indicative of at least the sterility assurance level or a lower level or no more signals from the biological indicator 14, successful sterilization is verified and the reader 44 may communicate to the controller 22 to stop the sterilization cycle. In such an embodiment, the controller 22 may continue the sterilization cycle until it receives a signal from the reader 44 to stop.

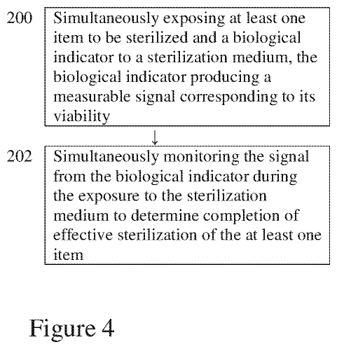

FIG. 4 shows a flow chart of an exemplary method of use of the system to monitor sterilization according to an aspect of the present invention. At least one item to be sterilized and a biological indicator are simultaneously exposed to a sterilization medium 200. The biological indicator used is an indicator which exhibits a readable signal indicative of viability of the spores in the indicator as previously described. The signal from the biological indicator is monitored simultaneously during the exposure of the at least one item being sterilized and the biological indicator to the sterilization medium in order to determine completion of effective sterilization 202. In such a way, the sterilization may be monitored real time.

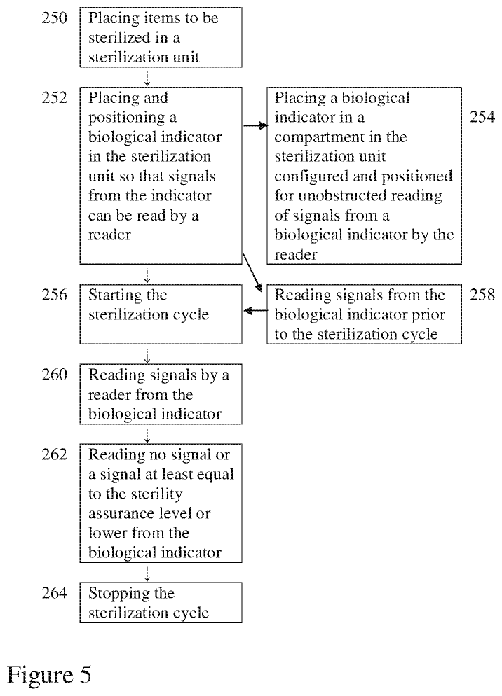

FIG. 5 shows a flow chart of an exemplary method of monitoring sterilization. FIG. 5 shows exemplary steps for monitoring sterilization according to the method described in FIG. 4. A sterilization unit, such as, but not limited to an autoclave is prepared for use. Items to be sterilized are placed in the sterilization unit 250. A biological indicator, which exhibits a readable signal indicative of viability of the spores in the indicator as previously described is placed in the sterilization unit 252. The biological indicator is positioned so that it will be exposed to substantially the same sterilization conditions as the items to be sterilized. The biological indicator is placed so that the signal from the indicator is readable by the reader. In some embodiments, the biological indicator is placed in a compartment in the sterilization unit, which is configured and positioned for unobstructed reading of signals from a biological indicator by the reader 254. In one embodiment the reader is connected to the autoclave such that it can read signals from the biological indicator as described hereinabove. In an alternative embodiment the reader is remote from the autoclave, but positioned so that it can read viability signals from the biological indicator. The sterilization cycle is started, the sterilization using any suitable sterilization medium and conditions 256. The reader may read signals from the biological indicator prior to the sterilization cycle 258. This pre sterilization reading may ascertain a baseline reading. The pre sterilization reading may verify that the biological indicator and reader are working. In some embodiments, the biological indicator exhibits luminosity or bioluminosity. The luminescence or bioluminocity signal may be read optically by an optical reader. One non-limiting example of a biological indicator used in the present invention is one which has been manipulated to exhibit a fluorescent signal, which is readable during the sterilization process. The fluorescent signal correlates with the degree of sterilization of items being sterilized in the sterilization unit for real time monitoring of the sterilization process. In some embodiments, the biological indicator may exhibit magnetism. A magnetic signal may be read by a magnetic reader. In some embodiments, live spores exhibit potential difference and the signal from the microorganism may be potential difference, which may be read by a voltmeter reader. The system may have been calibrated and adjusted for background radiation or other types of signals. A positive reading may be indicative that the microorganisms are alive. Dead microorganisms may not exhibit luminosity, bioluminosity, magnetic and potential difference signals. The reader may continually detect and read the signals from the biological indicator 260. In some embodiments, the reader may detect and read signals at certain time intervals, which may be predetermined. The microorganisms are configured such that they do not exhibit the signal being measured when they are dead. When the reader fails to detect and read a signal, or detects a signal indicative of at least the sterility assurance level or lower, the microorganisms are no longer viable and are dead, or a sufficient amount of the microorganisms are dead, for verification of sterility 262. The sterilization cycle may be stopped as the items in the sterilization unit are sterile 264. The order of the steps of the method is not meant to be limiting and may be in any suitable order.

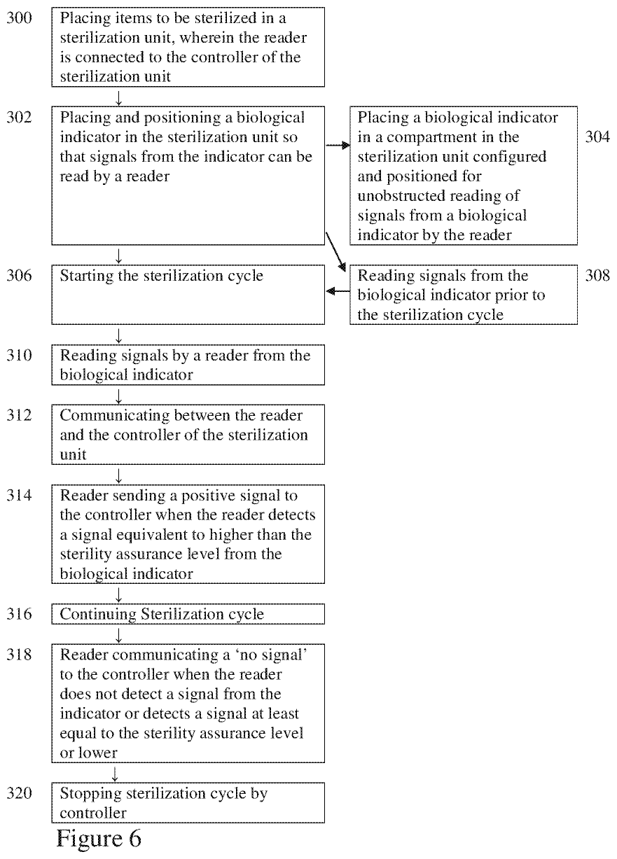

FIG. 6 shows a flow chart of an exemplary method of automated sterilization cycle time using a sterilization indicator according to an aspect of the present invention. In order to avoid repetition, steps 300-310 are as previously described for steps 250-260 in FIG. 5. However, in a method of automatic sterilization cycle time, a system of the present invention as described in FIG. 3 may be employed, wherein the reader is connected to the controller of the sterilization unit. The controller may communicate with the reader and vice versa 312. The reader may send a pre-sterilization baseline viability signal from the biological indicator to the controller. When the reader detects a signal (wherein the signal is equivalent to higher than the sterility assurance level of microorganisms) from the biological indicator, a positive signal is communicated to the controller 314 and the sterilization cycle continues 316. In some embodiments, according to the strength of the signal, the controller may change the sterilization conditions, which may affect the time of the sterilization cycle. When the reader no longer detects a signal from the biological indicator, or detects a signal at least indicative of the sterility assurance level or lower, the reader communicates a no ‘signal’ reading to the controller 318. As a result, the controller stops the sterilization cycle 320. In some embodiments, if no pre-sterilization signal is detected by the reader, it is an indication that something is wrong with the setup of the system or the biological indicator. In such an embodiment, no signal may be communicated to the controller and the controller will not start the sterilization cycle. The order of the steps of the method is not meant to be limiting and may be in any suitable order.

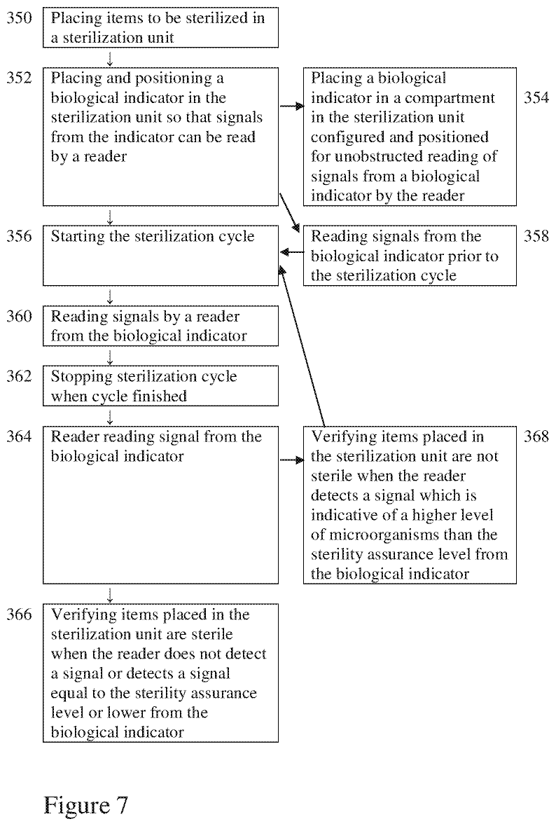

FIG. 7 shows a flow chart of an exemplary method of use of the system to verify sterilization according to an aspect of the present invention. A system of the present invention as described in FIG. 1 or FIG. 2 may be employed. In order to avoid repetition, steps 350-358 are as previously described for steps 250-258 in FIG. 5. In this method, signals from the biological indicator may be optionally read by a reader during the sterilization cycle 360. In the verification method, the sterilization cycle is stopped after the sterilization cycle has finished 362. The reader reads a signal from the biological indicator 364. If the reader does not detect a signal, or detects a signal indicative of the sterility assurance level or lower, the sterilization cycle is verified as being successful and the items placed in the sterilization unit are verified as sterile 366. If the reader detects and reads a signal from the biological indicator, where the signal is indicative of a higher level of microorganisms than the sterility assurance level, the sterilization cycle is proved incomplete and the items placed in the sterilization unit are unsterile 368. As a result, the sterilization cycle is resumed 356 until the process is verified as successful. The order of the steps of the method is not meant to be limiting and may be in any suitable order.

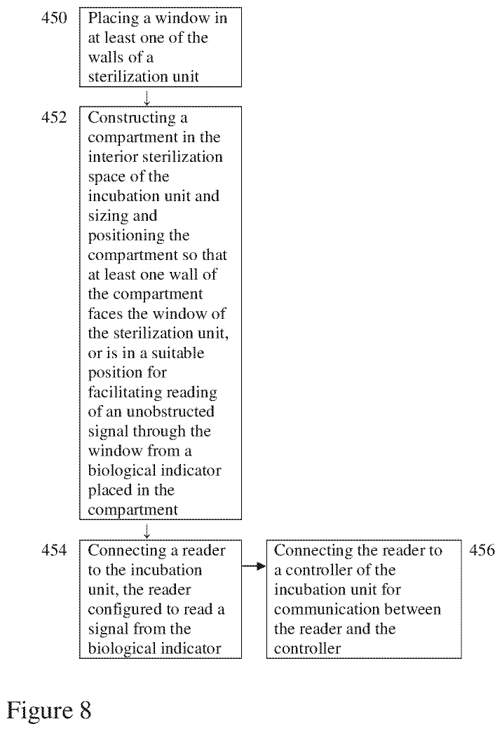

FIG. 8 shows a flow chart of an exemplary method of production of a system according to an aspect of the present invention. An incubation unit, such as an autoclave is constructed. The autoclave may include any standard features and non-standard features known in the art. The autoclave may include a controller to control the sterilization cycle. The autoclave may be made from a metallic material, which can withstand the sterilization conditions. The autoclave may include a cage for eliminating noise that could interfere with a reading by the reader of a signal from a biological indicator. A window is constructed in one of the walls of the incubation unit 450. The window is made of any suitable material through which a readable signal from a biological indicator can be read. In one embodiment, the window is on the ceiling of the incubation unit and the window is made from glass. The glass may be configured to withstand the sterilization conditions. A compartment for the biological indicator is constructed in the internal chamber of the incubation unit 452. The compartment is sized and positioned so that at least one wall of the compartment faces the window, or is in a suitable position for facilitating reading of an unobstructed signal from the biological indicator through the window. A reader configured to read a signal from the biological indicator is connected to the incubation unit 454. Connection may be by any suitable connection means, including but not limited to mechanical connection, electrical connection and a combination thereof. The reader includes a sensor, such as at least one of an optical sensor, an electrical sensor and a magnetic sensor. The reader may be calibrated, or may include calibration data, for reading signals from the biological indicator. The reader is positioned so that it is adjacent to the window and so that the sensor in the reader can detect a signal from the biological indicator through the window. The signal from the biological indicator may be at least one of a luminescent signal, an electric signal, a magnetic signal, a radiation signal, a voltage signal, a current signal, a fluorescent signal and a colorimeter signal. In one non-limiting example, the reader is connected to the ceiling of the incubation unit, wherein the ceiling of the incubation unit features a glass window, under which is a compartment in which the biological indicator is placed. In one embodiment, the reader is connected to a controller on the incubation unit 456. The reader is connected so that it can communicate to the controller and vice versa. The order of the steps of the method is not meant to be limiting and may be in any suitable order.

Reference is made to the following example, which together with the above descriptions illustrate the invention in a non-limiting fashion.

### EXAMPLE 1—Real Time Monitoring of a Sterilization Process

A biological indicator featuring spores of Geobacillus Stearothermophilus are added to a paper carrier and suitably packaged to maintain the viability of the combination and permit exposure of the spores to the sterilization medium and conditions. The spores have been manipulated to enable luminescence of the viable spores. The biological indicator is placed in an autoclave with a glass window. The biological indicator is put in a compartment facing the window of the autoclave. Surgical instruments to be sterilized are placed on a bottom shelf of the autoclave. A reader attached to the autoclave and positioned over the window so that it can read signals through the window reads a signal from the biological indicator. A positive signal pre sterilization is indicative that the microorganisms are alive and that the biological indicator reader system is working. The sterilization cycle is started and the reader detects and reads the signals from the biological indicator. The reader may translate the reading of the signal to an amount of viable microorganisms present in the biological indicator. When the reader no longer detects a signal or detects a signal indicative of the sterility assurance level or lower, the biological indicator is no longer substantially viable, indicative that the microorganisms are dead or a sufficient amount of the microorganisms are dead and that the sterilization cycle has been completed successfully. The sterilization cycle can then be stopped.

One skilled in the art can appreciate from the foregoing description that the broad systems, devices and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

What is claimed is:

1. A system for monitoring a sterilization procedure, the system comprising:
a sterilization unit for providing sterilization of at least one item;
a biological indicator for producing a readable signal, the signal readable during the sterilization procedure and the signal corresponding to viability of the biological indicator; and
a reader for reading the signal from the biological indicator during the sterilization procedure for monitoring the sterilization real time. The system of claim 1, wherein the sterilization unit comprises a window, through which the signal can be detected and read by the reader.

3. The system of claim 2, wherein the reader is adjacent to the window, for detecting and reading the signal from the biological indicator, through the window.

4. The system of claim 2, wherein the sterilization unit comprises a compartment for holding the biological indicator and wherein the compartment is constructed and positioned to facilitate reading of the signal from the biological indicator through the window.

5. The system of claim 1, wherein the biological indicator comprises a microorganism comprising at least one of Bacillus, Geobacillus and Clostridia species of microorganisms.

6. The system of claim 1, wherein the biological indicator produces a readable signal relating to at least one of chemiluminogenicity, bioluminogenicity, fluorogenicity, chromogenicity, electrochemiluminescence, photoluminescence, phosphorescence, thermoluminescence, potential difference, magnetism and radiation.

7. The system of claim 5, wherein the microorganism has been manipulated to exhibit bioluminescence.

8. The system of claim 1, wherein a measured signal, which is higher than a signal equivalent to a sterility assurance level indicates that the sterilization of the at least one item is not complete and wherein at least one of no signal, a signal equal to about the sterility assurance level and a signal equal to an amount of viable microorganisms lower than the sterility assurance level, indicates that the sterilization of the at least one item is complete.

9. The system of claim 1, wherein the sterilization unit is an autoclave.

10. The system of claim 1, wherein the reader comprises a sensor and wherein the sensor is at least one of an optical sensor, an electrical sensor and a magnetic sensor.

11. The system of claim 1, wherein the reader comprises a display for displaying the progress of a sterilization procedure.

12. The system of claim 1, wherein the sterilization unit comprises a controller for controlling the sterilization procedure and wherein the controller turns off the sterilization cycle as a result of the reader receiving and reading no signal or a signal equal to about the sterility assurance level or lower from the biological indicator.

13. The system of claim 1, wherein the biological indicator is covered by a substrate and wherein the substrate is permeable to the signal.

14. A method of monitoring sterilization, comprising:
simultaneously exposing at least one item to be sterilized and a biological indicator to a sterilization medium, the biological indicator producing a measurable signal corresponding to its viability; and
simultaneously monitoring the signal from the biological indicator during the exposure to the sterilization medium to determine completion of effective sterilization of the at least one item.

15. The method of claim 14, wherein exposing comprises:
placing the biological indicator comprising a microorganism in a sterilization unit of the system of claim 1, the sterilization unit containing the at least one item to be sterilized; and
running a sterilization cycle in the sterilization unit; and
wherein monitoring comprises;
reading a signal from the biological indicator during the sterilization cycle.

16. The method of claim 15, wherein placing comprises placing the biological indicator so that it is readable by the reader through a window in the sterilization unit.

17. The method of claim 15, further comprising reading a signal from the biological indicator prior to the start of the sterilization cycle.

18. The method of claim 15, wherein reading a signal, comprises reading at least one of luminosity, a magnetic signal and potential difference from the biological indicator.

19. The method of claim 15 further comprising:
stopping the sterilization process according to the determining from the signal when the sterilization is successfully completed, wherein stopping the sterilization process comprises:
the reader communicating with a sterilization unit controller when the reader detects no signal from the biological indicator or a signal equal to or lower than about the sterility assurance level; and
the controller turning off the sterilization process as a result of the communication of no signal or a signal equal to about or lower than the sterility assurance level.

20. A sterilization device for monitoring a sterilization procedure, comprising:
a sterilization unit for providing sterilization of at least one item, the sterilization unit comprising:
an internal chamber for placing the at least one item to be sterilized;
a compartment in the internal chamber for holding a biological indicator, the biological indicator producing a readable signal, the signal corresponding to viability of the biological indicator; and
a window, through which a signal from the biological indicator can be detected and read by a reader; and
a reader connected to the sterilization unit, the reader for reading the signal from the biological indicator, wherein the reader is adjacent to the window, for detecting and reading the signal from the biological indicator through the window.
